(19) United States
(12) Patent Application Publication
(10) Pub. No.: US 2020/0126653 A1
(43) Pub. Date: Apr. 23, 2020

(54) METHOD AND SYSTEM FOR MONITORING AND DISPENSING CONTROLLED SUBSTANCES

(71) Applicant: David Seltzer, Granite City, IL (US)

(72) Inventor: David Seltzer, Granite City, IL (US)

(21) Appl. No.: 16/661,788

(22) Filed: Oct. 23, 2019

Related U.S. Application Data
(63) Continuation-in-part of application No. 16/056,099, filed on Aug. 6, 2018.

(51) Int. Cl. G16H 20/13; G16H 10/60; G07F 17/00; G06F 21/31

(52) U.S. Cl. CPC G16H 20/13; G06F 21/31; G07F 17/0092; G16H 10/60

(57) ABSTRACT

A method of monitoring and dispensing controlled substances includes providing a machine containing a plurality of controlled substances. Each of the controlled substances is in the machine in a separate vessel. The machine is connected with a network having an accessible patient information database. The machine receives, from the network, an order for a specific patient having a specific patient record in the patient information database for a prescribed amount of one of the controlled substances. The machine dispenses only the prescribed amount of the one controlled substance to a container from one of the separate vessels. The machine or the network permits an authorized user to retrieve the container having the dispensed prescribed amount of the one controlled substance from the machine to fill the order. The process is repeated when a new order is received by the machine from the network for any one of the controlled substances.

### RELATED APPLICATION DATA

This patent is a continuation-in-part application entitled to the benefit of and claims priority to co-pending U.S. Non-Provisional application Ser. No. 16/056,099 filed Aug. 6, 2018 and entitled “Method for Monitoring and Dispensing Controlled Substances.” The entire contents of this prior filed application are hereby incorporated herein by reference.

### BACKGROUND

### 1. Field of the Disclosure

The present disclosure is generally directed to a method and a system of monitoring and dispensing controlled substances, and more particularly, to a method and system that inhibit distribution of excess amounts of controlled substances and diversion of such excess amounts to unauthorized users.

### 2. Description of Related Art

Opioid overdose has become a major concern in the recent past. Misuse of and addiction to opioids has a significant impact on public health and also significantly impacts social and economic welfare. As one attempt at a solution to the epidemic, the U.S. government has recently significantly reduced overall opioid production. Reduced production of opioids may have the negative result of creating a shortage of medically necessary or useful opioids. Thus, monitoring the distribution of available opioids can be vital to ensure that all patients are supplied with the required medication and the subscribed amount of the medication.

Existing opioid distribution methods and systems utilize stored medical records that are retrievable electronically and local dispensing machines that can dispense prepackaged quantities of drugs and medications. One such example of a patient record system or patient database is maintained by Epic Systems Corporation. The Epic Systems database is said to hold more than half of all patient records in the United States. One such example of a dispensing machine is a Pyxis machine provided by the Pyxis Corporation. Pyxis machines are located, for example, on site in hospitals. Hospitals often have multiple Pyxis machines within a single hospital, which can each be provided to serve specific floors, dispense only specific drugs, or the like. Physicians can prescribe specific drugs in specific quantities for hospital patients. The physicians can send a prescription order via a network to a specific hospital or Pyxis machine for a specific patient. Authorized medical staff at the hospital can be notified that a prescription for a specific drug and quantity can be retrieved from the Pyxis machine. An authorized person can access the prescribed drug from the machine by entering an access code.

The typical system provides such machines with prepackaged quantities of drugs or medications. Such packages may be provided in the dispensing machine in, for example, 2 milliliter (ml) and 10 ml vials. When a physician places an order for a specific drug in a quantity that can be created exactly by either one of the prepackaged amounts or a combination of two or more of the prepackaged amounts, problems may be avoided. For example, an order may require dispensing 2 ml, 4 ml, 10 ml, 12 ml, or 20 ml of a drug. These quantities can be dispensed in the exact amount required using one or a combination of two or more of the prepackaged vials. When a physician places an order for a quantity that cannot be dispensed in the exact amount, the existing system dispenses an excess amount of the drug. For example, an order may require 3 ml, which can be met by dispensing two of the 2 ml vials but leaves an excess amount of the drug of 1 ml. In another example, an order may require 13 ml, which can be met by dispensing one 10 ml vial and two 2 ml vials or by dispensing two 10 ml vials. In either case, an excess amount, either 1 ml or 7 ml, of the drug is dispensed.

The excess amounts must be discarded and cannot be re-used or saved for later use. This can result in excessive and unnecessary waste of the dispensed drugs. This can also result in increased manufacturing cost of the drugs. Further, it is typically required that the authorized medical person have a witness, i.e., a second authorized person, witness the act of discarding the excess drug amounts and corroborate the act in the record. This can waste valuable time of the medical staff, which is better spent working with patients than in discarding unused dispensed drugs. These excess amounts are also potentially vulnerable, instead of being properly discarded, to being obtained and/or distributed by unauthorized personnel and/or to people with no authorization or prescription.

With current opioid distribution systems, the medical staff issuing the controlled substances must complete the related documentation or record update process as well. Therefore, in addition to caring for the patients, the medical staff also needs to document or record the dosages given to each patient, update the medical information for each patient, and the like. By having to complete the documentation or recording procedures during the time allocated for the patient, the overall time that the medical professionals can devote to the patients is significantly reduced.

### SUMMARY

In one example, according to the teachings of the present disclosure, a method is disclosed for monitoring and dispensing controlled substances. The method includes providing a machine containing a plurality of controlled substances, wherein each of the plurality of controlled substances stored in the machine in separate vessels. The machine is connected with a network having an accessible patient information database. The method also includes receiving, by the machine from the network, an order for a specific patient having a specific patient record in the patient information database for a prescribed amount of one controlled substance of the plurality of controlled substances. The method also includes dispensing only the prescribed amount of the one controlled substance to a container from a respective vessel of the separate vessels. The method also includes permitting an authorized user to retrieve the container having the dispensed prescribed amount of the one controlled substance from the machine to fill the order. The method also includes repeating each of the steps when a new order is received by the machine from the network for any one of the plurality of controlled substances.

In one example, the step of dispensing can include dispensing the prescribed amount of the one controlled substance into a syringe as the container.

In one example, the step of providing can include providing the machine having a control device with a processor and a memory. The control device can be configured to determine a current volume of the one controlled substance before the step of dispensing and can be configured to determine a new current volume of the one controlled substance after the step of dispensing. The new current volume becoming the current volume or a next or subsequent current volume before a subsequent step of dispensing the one controlled substance.

In one example, the method can further include storing a threshold refill level for a corresponding substance of the plurality of controlled substances for each respective vessel of the separate vessels. The method can also include monitoring the new current volume of each of the separate vessels and comparing the new current volume of each respective vessel of the separate vessels to the respective threshold refill level for each corresponding substance of the plurality of controlled substances. The method can also include notifying the authorized user if the new current volume is less than the threshold refill level for any one vessel of the separate vessels. The method can also include prompting the notified authorized user to refill the one vessel with the corresponding substance. The notifying and prompting steps can be different or can be one and the same.

In one example, the method can include providing or including an authorized user account for an authorized user. The authorized user account can be managed by an external server of the network.

In one example, the step of dispensing can further includes transferring the prescribed amount of the one controlled substance from the respective vessel to the container in a filling station of the dispensing unit.

In one example, the method can include providing a plurality of pipettes in a filling station. Each pipette can be arranged to cooperate with one of the separate vessels for transferring a corresponding one of the plurality of controlled substances.

In one example, the method can further include applying a label to the container. The label can include patient information that at least identifies the specific patient.

In one example, the method can further include applying a label to the container. The label can include substance information that at least identifies the one controlled substance and the prescribed amount.

In one example, each of the plurality of controlled substances can be selected from a group consisting of Hydromorphone, Methadone, Meperidine, Oxycodone, Morphine, Fentanyl, and Codeine.

In one example, the method can further include sending a notification to the authorized user through the network or the machine when the order is received by the machine. The method can also include prompting the authorized user to remove the container having the prescribed amount of the one controlled substance from the machine.

In one example, the specific patient record can include patient identification information, prescribed controlled substance information, and dosage information for each prescribed controlled substance. The method can also include sending the patient identification information, prescribed controlled substance information, and dosage information of the specific patient record to the machine and updating the dosage information for the specific patient record after the one controlled substance is dispensed.

In one example according to the teachings of the present disclosure, a system is disclosed for monitoring and dispensing controlled substances. The system includes a machine containing a plurality of controlled substances. Each of the plurality of controlled substances is stored in the machine in separate vessels. A network is connected to the machine and has an accessible patient information database. The machine is configured to receive, from the network, an order for a specific patient from the patient information database for a prescribed amount of one controlled substance of the plurality of controlled substances. The machine is configured to, when the order is received, dispense only the prescribed amount of the one controlled substance to a container from a corresponding vessel of the separate vessels. A user interface of the machine is configured to permit an authorized user to obtain access to and retrieve the container having the dispensed specific amount of the one controlled substance from the machine.

In one example, the prescribed amount of the one controlled substance can be dispensed into a syringe as the container.

In one example, the machine can have a control device with a processor and a memory. The control device can be configured to determine a current volume of the one controlled substance before dispensing any of the one controlled substance. The control device can also be configured to determine a new current volume of the one controlled substance after dispensing. The new current volume can become the current volume, i.e., the next current volume or subsequent current volume, before a next subsequent dispensing of the one controlled substance.

In one example, the separate vessels can be refillable, replaceable, or refillable and replaceable within the machine. The network or the machine can detect when the new current level of each of the plurality of controlled substances reaches a threshold level within the corresponding separate vessels.

In one example, the machine or the network can notify an authorized user if the new current volume is less than the threshold refill level for any one vessel of the separate vessels, and/or can prompt the notified authorized user to refill the one vessel with the corresponding substance.

In one example, the machine can transfer the prescribed amount of the one controlled substance from the respective vessel to the container in a filling station of the dispensing unit. The filling station can include a plurality of pipettes and each pipette can be arranged to cooperate with one of the separate vessels for transferring a corresponding one of the plurality of controlled substances.

In one example, the machine can apply a label to the container. The label can include patient information that at least identifies the specific patient and at least identifies the one controlled substance and the prescribed amount.

In one example, each of the plurality of controlled substances for the system can be selected from a group consisting of Hydromorphone, Methadone, Meperidine, Oxycodone, Morphine, Fentanyl, and Codeine.

### BRIEF DESCRIPTION OF THE DRAWINGS

The drawings provided herewith illustrate one or more examples or embodiments of the disclosure and therefore should not be considered as limiting the scope of the disclosure. There may be other examples and embodiments that may be equally effective to achieve the objectives and that may fall within the scope of the disclosure. Objects, features, and advantages of the present invention will become apparent upon reading the following description in conjunction with the drawing figures, in which:

FIG. 1 shows a generic schematic illustration of a machine for dispensing controlled substances and constructed in accordance with the teachings of the present disclosure.

FIG. 2 shows a system, including the machine of FIG. 1, and in accordance with the teachings of the present disclosure.

FIG. 3 shows a flow chart of one example of a method in accordance with the teachings of the present disclosure.

FIG. 4A is a flowchart illustrating the basic overall process of the present invention, and

FIG. 4B is a continuation thereof, further illustrating the basic overall process of the present invention.

FIG. 5 is a flowchart illustrating the basic overall process of refilling the substance storage unit.

FIG. 6 is a flowchart illustrating the basic overall process of measuring the new level of the specific controlled substance by weight.

FIG. 7 is a flowchart illustrating the basic overall process of measuring the new level of the specific controlled substance by volume.

FIG. 8 is a flowchart illustrating the basic overall process of retrieving the volume of the specific controlled substance.

FIG. 9 is a flowchart illustrating the basic overall process of transferring the patient identification and updating the dosage information for the specific medical record.

FIG. 10 is a flowchart illustrating the basic overall process of using a plurality of pipettes in the dispensing unit.

FIG. 11 is a flowchart illustrating the basic overall process of using a plurality of syringes in the labelling unit.

The use of the same reference numbers or characters throughout the description and drawings indicates similar or identical components, aspects, and features of the disclosure.

### DETAILED DESCRIPTION OF THE DISCLOSURE

The objective of the present disclosure is to address the aforementioned issues. In particular, the present disclosure introduces a method and a system to monitor the distribution and the use of controlled substances such that the waste and abuse of controlled substances is eliminated. Additionally, the present disclosure can help to ensure that only the prescribed amounts of a certain medication is dispensed from a machine and given to a patient.

All illustrations in the drawings are for the purpose of describing selected versions of the present disclosure and are not intended to limit the scope of the present disclosure. Several embodiments of the disclosure are also described and shown herein. These embodiments are not intended to limit the scope of the disclosure or claims to only those embodiments and combinations of steps or elements. Each element of the described systems and step of the described methods may be employed separately or in other different combinations of the elements and steps.

The present disclosure introduces methods and systems that can monitor the use and the distribution of controlled substances. The methods and systems of the present disclosure are intended to be used with intravenous schedule II drugs or other comparable controlled substances. Since the monitoring and the distribution process introduced by the present disclosure is automated, the need to manually document controlled substance related data is eliminated. Additionally, since the methods and systems of the present disclosure are designed to dispense an exact prescribed amount of a controlled substance, the overall waste of the controlled substance is also eliminated. Since an excess of the controlled substance is not released to the patient, the overall possibility of the controlled substance being misused or redirected in an unauthorized manner is substantially reduced.

To execute the intended functionalities of the disclosed systems and methods, the present disclosure is provided with a controlled substance storage unit or device, i.e., a dispensing machine or machine, as described herein. The machine is connected to an external network, which may have an external server and/or a database that stores a plurality of medical records, which is managed by the external server. A specific controlled substance that a patient may require may be monitored and distributed according to the information stored in a specific patient record of the stored medical records.

To provide the specific controlled substance as required, the machine of the present disclosure stores a plurality of the controlled substances. Each of the plurality of controlled substances is provided in the machine in a current quantity, volume, or amount within a separate vessel. The machine is configured to record the initial volume of each of the plurality of controlled substances and to monitor the changing volume or amount as the controlled substance is dispensed over time.

The systems and methods of the present disclosure are intended to be used with intravenous, i.e., liquid form, schedule II drugs. Therefore, the plurality of controlled substances stored in the substance storage unit or machine and dispensed according to the disclosed methods can be selected from a group comprising, but not limited to, Hydromorphone, Methadone, Meperidine, Oxycodone, Morphine, Fentanyl, and Codeine. However, the present disclosure can also be compatible for use with other controlled substances as well.

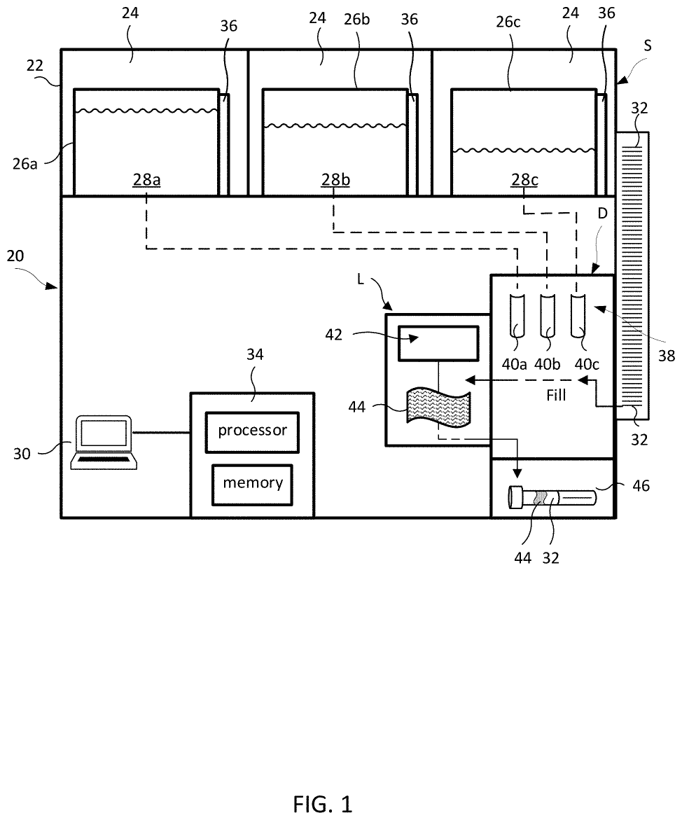

In one example according to the teachings of the present disclosure, as shown in FIG. 1, the methods and systems described herein utilize a dispensing machine, i.e., a machine 20. The size and shape of the machine 20, as well as many of the structural and functional details of the machine, can vary. Thus, in the drawings, the machine 20 is merely depicted in a simplified schematic form. In one example, the machine 20 can have a secure shell or housing 22, such as a sturdy metal structure, that houses separate units of the machine. In one example, the machine 20 has a substance storage unit S, which may include a set of distinct sub-compartments 24. The set of sub-compartments 24 is used and configured to hold a corresponding plurality of vessels 26, which respectively contain a plurality of the controlled substances 28. The vessels and controlled substances are collectively referred to herein using 26 and 28, respectively. As an example, as depicted in FIG. 1, if three specific and/or different controlled substances 28a, 28b, and 28c are held within the substance storage unit S, the three controlled substances may be held in three separate sub-compartments 24 in three separate vessels 26a, 26b, and 26c within the substance storage unit. However, the machine 20 may include fewer or more than the three vessels 26 and controlled substances 28 of the illustrated example.

In one example, the machine 20 also has a user interface 30 accessible on the shell or housing 22. The user interface 30 can be configured to permit an authorized user to obtain access to and retrieve a container 32, from a plurality or store (see FIG. 1) of such containers, that holds a dispensed specific amount of a selected one of the controlled substances 28 from the machine 20. The user interface 30 can be a touch screen, a keyboard, a monitor or display, any combination thereof, or any other suitable interface, mechanism, or device that permits an authorized user to interact with the machine 20.

In one example, the machine 20 can have a control device 34, such as a controller or control unit, that includes a processor and a memory. The control device 34 can include a printed circuit board (PCB), which may carry the processor and the memory. The control device 34 can communicate with the user interface 30, allowing an authorized user to operate, access, and manipulate the control device, as needed, through the user interface. The machine 20 can also include a sensor 36 associated with each of the vessels 26. The sensors 36 may be any type of liquid level sensor (liquid height, volume, weight, etc.) and can be configured to determine a current volume of the controlled substance in each corresponding vessel 26. In other examples, the control device, or a portion thereof, may be remote from the machine, such as a handheld electronic device held by an authorized user and configured to communicate with the machine.

In one example, the machine 20 can also include a dispensing unit D that includes a filling station 38, which can be configured to transfer a prescribed amount of a selected one of the controlled substances 28 from the respective vessel 26 to one of the containers 32. The filling station 38 can include, collectively, a plurality of pipettes 40. Each pipette 40a, 40b, and 40c can be arranged to cooperate with a designated one of the separate vessels 26a, 26b, and 26c. The pipettes 40a, 40, or 40c can be configured to transfer a prescribed amount of a corresponding one of the plurality of controlled substances 28a, 28b, and 28c from the vessel to a container 32. The dispensing unit D may also include a retrieval station or access point 46 that is accessible from outside the housing or shell 22. The retrieval station or access point 46 may have a holding compartment within the machine 20 for holding the container 32, filled with a prescribed amount of one of the controlled substances 28, to be retrieved by an authorized user. The retrieval station 46 may also have an access door (not shown) that is controlled to be ordinarily closed and locked. The access door may be operable through the user interface 30 and the control device 34 so that it is openable only by an authorized user.

In one example, the machine 20 can also include a labeling unit or station L in the shell or housing 22. The labeling unit or station L can be configured to hold a plurality of blank labels or labelling material. The labeling unit or station L can also be configured with printing equipment or devices 42 to print specific information onto a single label 44 and to apply the printed label to the container 32. The label 44 can include patient information that at least identifies the specific patient and substance information that at least identifies the one controlled substance within the container 32 and the prescribed amount within the container.

The specific features, components, and functions of the machine 20, the housing or shell 22, the storage unit S, the dispensing unit D, the labeling unit L, the controller or control device 34, and the user interface 30 can vary. The various aspects of the machine 20 can include known, existing components or yet-to-be developed components. The various aspects of the machine 20 can also function according to known, existing methods or processes or yet-to-be developed methods or processes.

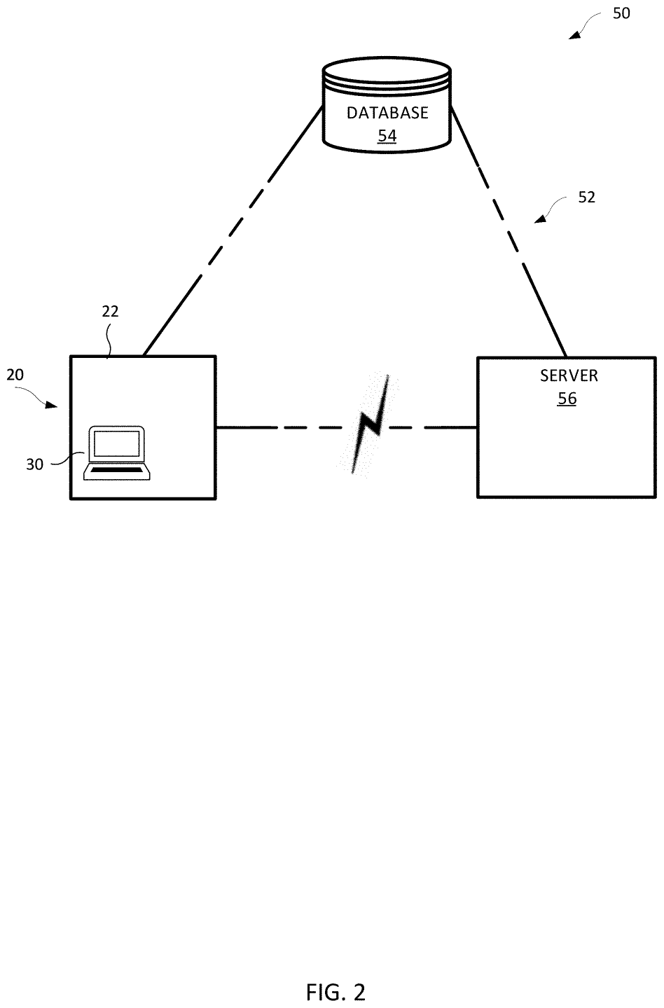

In one example according to the teachings of the present disclosure, a system 50, as shown in FIG. 2, is disclosed for monitoring and dispensing the controlled substances 28. The system 50 includes the machine 20 containing the plurality of controlled substances 28. Each of the plurality of controlled substances 28 is stored in the 20 machine in the separate vessels 26. The machine 20 is connected to a network 52, which has an accessible patient information database 54. The network 52 may be wire connected to the machine 20 or wirelessly connected to the machine. The network 52 may include a dedicated server 56 or multiple servers (not shown) that include and store, or that are connected to, the patient information database 54. The patient information database 54 may instead be accessible from a storage in the cloud.

The machine 20 of the system 50 is configured to receive, from the network 52, an order for a specific patient from the patient information database 54. The order can come from the user interface 30 of the machine 20, which is also considered as a part of the network 52. The order can instead come from another part of the network 52 in the same facility as the machine 20 or a different facility. The order can come from a remote device, such as a handheld electronic device, i.e., an electronic tablet or note pad, a smartphone, or the like, which is connected to the network 52. The order can be for a prescribed amount of one of the controlled substances 28a, 28b, or 28c of the plurality of controlled substances 28.

The machine 20 of the system 50 is configured to, when the order is received, dispense only the prescribed amount of the one controlled substance 28a, 28b, or 28c to a container 32 from a corresponding vessel 26a, 26b, or 26c of the separate vessels 26. The user interface 30 of the machine 20 is configured to permit an authorized user to obtain access to and retrieve the container 32 having the dispensed specific amount of the one controlled substance 28a, 28b, or 28c from the machine. In one example, the prescribed amount of the one controlled substance 28a, 28b, or 28c can be dispensed into a syringe as the container 32. In other examples, the container 32 can be a bottle, a vial, or another type of suitable container.

In one example, the processor and memory of the control device 34 of the machine 20 can be configured to determine a current volume of the one controlled substance 28a, 28b, or 28c within its respective vessel 26a, 26b, or 26c before each occurrence of dispensing any of the one controlled substance. The processor and memory can also be configured to determine a new current volume of the one controlled substance after each dispensing of the one controlled substance. The new current volume can become the current volume, i.e., the next current volume or subsequent current volume, before a next subsequent dispensing of the one controlled substance.

In one example, the separate vessels 26 can each be refillable, replaceable, or refillable and replaceable within the machine 20. The network 52, the machine 20, or both, can detect when the new current level of each of the plurality of controlled substances 28 reaches a threshold level within the corresponding separate vessels 26. The machine 20 can be configured to provide access to an authorized user for refilling or replacing any one or more of the vessels 26 as necessary. The control device 34, the network 52, or both, can be configured to track the volume of each vessel 26 and each refilling and replacement of the vessels 26 to accurately monitor the usage and dispensing of the controlled substances 28.

In one example, the machine 20, the network 52, or both, or the system 50 can notify an authorized user if the new current volume is less than the threshold refill level for any one vessel 26a, 26b, or 26c of the separate vessels 26. The machine 20, the network 52, or both, can also be configured to prompt the notified authorized user to refill the one vessel with the corresponding substance. The notification and prompt actions can be separate actions or can be one and the same.

In one example, the machine 20 of the system 50 can transfer the prescribed amount of the one controlled substance 28a, 28b, or 28c from the respective vessel 26a, 26b, or 26c to the container 32 in the filling station 38 of the dispensing unit D. In one example, the filling station 38 can include, collectively, the plurality of pipettes 40 (see FIG. 1). Each pipette 40 can be arranged to cooperate with a corresponding one of the separate vessels 26 for transferring a corresponding one of the plurality of controlled substances 28. The pipettes 40 can be moved and manipulated by a mechanism (not shown) in the filling station 38 to receive a dispensed amount of a controlled substance 28 and to dispense the dispensed amount to the selected container 32. The control device 34 may be configured to communicate with the mechanism to operate the pipettes 40, as needed to fill an order.

In one example, the machine 20 of the system 50 can apply one of the printed labels 44 to the container 32 within the labelling unit L. The machine 20 can be include the printing devices or mechanisms 42 in the labelling unit L. The labelling unit L may be a part of the filling station 38 or another part of the dispensing unit D, of can be a separate unit and area within the machine. The labelling unit L can select a label from a store of multiple unprinted labels, to print the information on the labels, to manipulate and move the printed label 44, and to apply the printed label to the container 32. The control device 34 may be configured to communicate with the label mechanisms 42 to operate the printing and other mechanisms, as needed to fill an order. The printed label 44 can include the patient information that at least identifies the specific patient and that at least identifies the one controlled substance 28a, 28b, or 28c and the prescribed amount of the substance. The machine 20, the network 52, or both, can be configured to record and store the information details. The patient information database 54 can be updated each time a container 32 is dispensed by the machine 20 to accurately track the patient information for each prescribed patient and each prescribed usage or dispensing of the controlled substances 28.

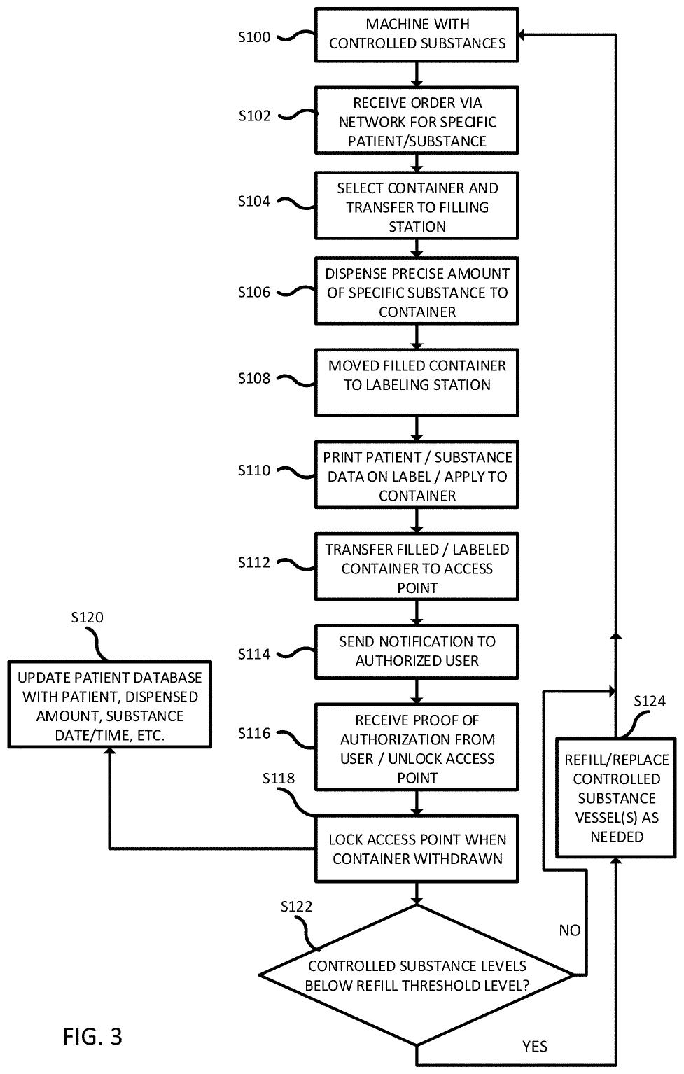

In one example, according to the teachings of the present disclosure, a method as shown in FIG. 3 is disclosed for monitoring and dispensing controlled substances. The method includes providing the machine 20 containing the plurality of controlled substances 28 (S100). Each of the plurality of controlled substances 28 is stored in the machine 20 in corresponding separate vessels 26. The machine 20 is connected with the network 52, which has the accessible patient information database 54.

The method also includes receiving, by the machine 20 from the network 52, an order for a specific patient having a specific patient record in the patient information database 54 (S102). The order can be placed by an authorized user through any part of the network 54 for a prescribed amount of one controlled substance 28a, 28b, or 28c of the plurality of controlled substances 28. The method can also include the machine 20 selecting one container 32 from a store of a plurality of the containers connected to or held within the machine (S104). The method also includes dispensing only the prescribed amount of the one controlled substance 28a, 28b, or 28c to the container 32 from a respective vessel 26a, 26b, or 26c of the separate vessels 26 (S106).

The method also includes permitting an authorized user to retrieve the container 32 having the dispensed prescribed amount of the one controlled substance 28a, 28b, or 28c from the machine 20 to fill the order (S116, S118). The method can include the authorized user accessing the container from the dispensing unit D after proof of authorized access (S116). Such proof can be entry of an identification (ID) or pass code into the user interface, sending of same via the network to the machine 20, electronic recognition technology (finger or thumb print, voice, face, retina, or other type of recognition), or the like. The method can include the dispensing unit D unlocking or opening an access door to a receiving or retrieval space in the machine, upon receipt of proof of authorization (S116). Once the container 32 is retrieved by the authorized user, the machine 20 can again close and lock the dispending unit D (S118).

The method also includes repeating each of the foregoing steps when a new order is received by the machine 20 from the network 52 for any one of the plurality of controlled substances 28 (S102 et. seq.). The method can also include updating the patient information database upon each dispending step (S120). The method can also include updating information corresponding to the amounts of the controlled substances 28 having been dispensed and remaining within the vessels 26.

In one example, the step of dispensing can include dispensing the prescribed amount of the one controlled substance into a syringe as the container 32. The authorized user can take the syringe, which has the exact prescribed amount of the controlled substance, to the patient or to the appropriate medical personnel to administer the controlled substance to the patient. The entire contents of the syringe can be administered to the patient, leaving no excess or waste amount of the controlled sub stance.

In one example, the step of providing can include providing the machine 20 having the control device 34 with a processor and a memory. The control device 34 can be configured or programmed to determine the current volume of the one controlled substance 28a, 28b, or 28c before the step of dispensing. The control device 34 can also be configured to determine a new current volume of the one controlled substance 28a, 28b, or 28c after the step of dispensing. The new current volume becoming the current volume or a next or subsequent current volume before a next or subsequent step of dispensing the one controlled substance 28a, 28b, or 28c.

In one example, the method can further include storing a threshold refill level for a corresponding substance 28a, 28b, or 28c of the plurality of controlled substances 28 for each respective vessel 26a, 26b, or 26c of the separate vessels 26 (S122). The method can also include monitoring the new current volume of each of the separate vessels and comparing the new current volume of each respective vessel of the separate vessels 26 to the respective threshold refill level for each corresponding substance of the plurality of controlled substances 28. The storing and monitoring steps can be carried out at the machine 20 level via the memory and processor of the control device 34, carried out at the network 52 level, or both.

The method can also include notifying the authorized user if the new current volume is less than the threshold refill level for any one vessel 26a, 26b, or 26c of the separate vessels 26 (S114). The notification can be provided as a visual notification, an audible notification, or both. The notification can be provided at the machine 20 via the user interface 30, a speaker, or both. The notification can be provided as a visual notification, an audible notification, or both, via a remote device such as an electronic tablet or note pad, a smart phone, or the like. The notification can be a text or message, an audible ping or custom sound, and/or the like. The method can also include prompting the notified authorized user to refill the one vessel with the corresponding substance. The prompt can be provided in the same various manner or manners as the notification. The notifying and prompting steps can be different or can be one and the same.

In one example, the method can include providing or including an authorized user account for each of one or more authorized users. Each authorized user account can be managed by an external server at the network 52 level, by the control device 34 at the machine 20 level, or both. The authorized user account can include the ID information noted above that allows the authorized user to access the network and the machine to be able to place an order and to retrieve the order from the machine.

In one example, the system 50 and the method may include having multiple machines 20 (not shown) as a part of the system and connected to the network 52. The multiple machines may be provided within the same facility, within multiple different facilities, or both.

In one example, the step of dispensing can further includes transferring the prescribed amount of the one controlled substance 28a, 28b, or 28c from the respective vessel 26a, 26b, or 26c to the container 32 in the filling station 38 of the dispensing unit D.

In one example, the method can include providing the plurality of pipettes 40 in the filling station 38 of the dispending unit D. Each pipette 40a, 40b, 40c can be arranged to cooperate with a corresponding one of the separate vessels 26a, 26b, and 26c for transferring a corresponding one of the plurality of controlled substances 28 form the vessel to the container 32 to be dispensed by the machine 20.

In one example, the method can further include moving the filled container 32 to the labeling unit L and applying the printed label 44 to the container 32 within the labelling unit L (S108, S110). The label 44 can include the patient information that at least identifies the specific patient. The label 44 can include substance information that at least identifies the one controlled substance and the prescribed amount. The method can also include transferring the filled and labeled container 32 to the retrieval station or access point 46 (S112).

In one example, the method can further include sending a notification to the authorized user through the network 52, the machine 20, or both, when the order is received by the machine 20. The method can also include prompting the authorized user to remove the container 32, having the prescribed amount of the one controlled substance, from the machine 20. The method can also include notifying the authorized user when the order is prepared and ready to be dispensed (S114). The notification and prompting steps can be conducted in the same manners as noted above.

In one example, each specific patient record in the patient information database 54 can include patient identification information, prescribed controlled substance information, and dosage information for each prescribed controlled substance. The method can also include sending the patient identification information, prescribed controlled substance information, and dosage information of the specific patient record to the machine 20 and updating the dosage information for the specific patient record after the one controlled substance 28a, 28b, or 28c is dispensed (S120).

In other examples, when the disclosed machine 20, system 50, network 52, and method are in use, a selection is received from a computing device, wherein the selection corresponds to a specific medical record selected from the plurality of medical records. Since the plurality of medical records is used to access the substance storage unit S of the machine 20, each of the plurality of medical records may comprise at least one controlled substance data. To select the specific medical record, the disclosed machine 20, system 50, network 52, and/or method may be provided with an authorized user account, which is managed by the external server. In one example, the authorized user account will be operated by a medical professional. The limited access to the plurality of medical records and the substance storage unit S is essential for safety purposes. Moreover, since the plurality of medical records is stored on the external server, the need to manually update each of the plurality of medical records is eliminated.

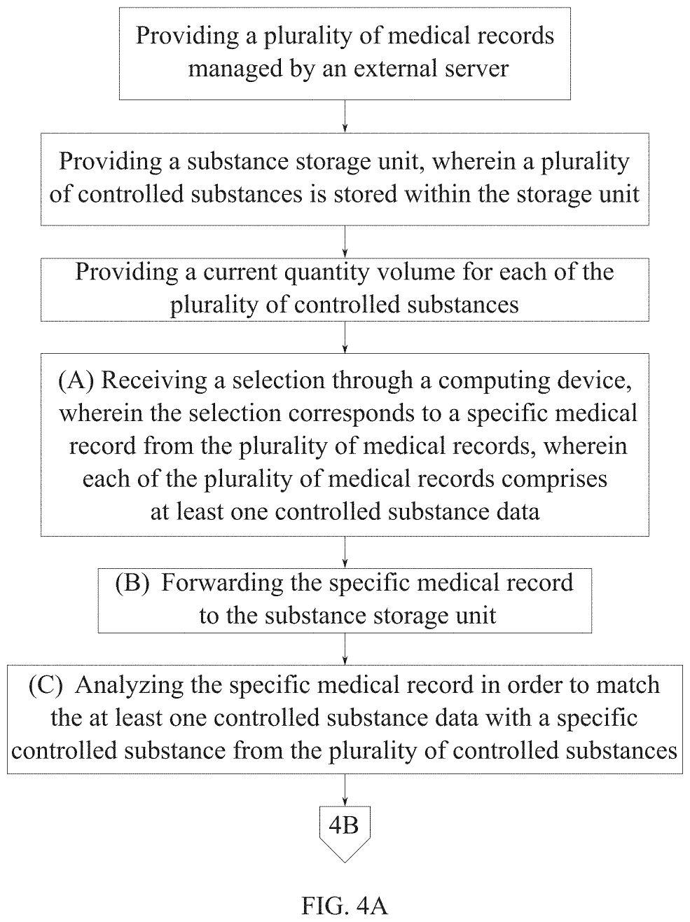

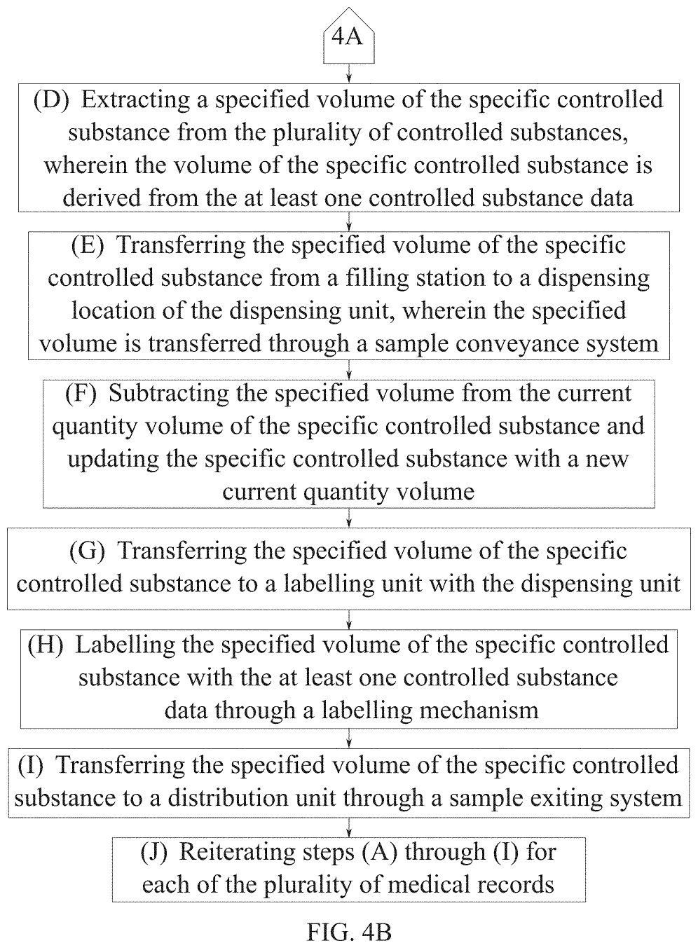

As illustrated in FIG. 4A, when the selection for the specific medical record is received through a computing device, the specific medical record is forwarded to the machine 20, including the substance storage unit S, so that the substance storage unit can initiate the controlled substance monitoring and distribution process. The machine 20, system 50, network 52, and/or method of the present disclosure may analyze the specific medical record in order to match the at least one controlled substance data with a specific controlled substance from the plurality of controlled substances. As an example, if the at least one controlled substance data requests Morphine, the machine 20, system 50, network 52, and/or method may match the specific medical record with the sub-compartment 24 and vessel 26 containing Morphine, wherein the sub-compartment is within the substance storage unit S. Upon matching the at least one controlled substance data with the specific controlled substance 28a, 28b, or 28c, the machine 20, system 50, and method may extract a specified volume of the specific controlled substance 28a, 28b, or 28c from the plurality of controlled substances 28, wherein the specified volume of the specific controlled substance is determined by the at least one controlled substance data. In reference to the previous example, if 5-milliliters (ml) of Morphine is required in the at least one controlled substance data of the specific medical record, 5-ml of Morphine is extracted from the sub-compartment 24 and vessel 26 storing Morphine. Since the exact amount of the specific controlled substance is extracted, the specific controlled substance is not wasted.

As illustrated in FIG. 4B, upon extracting the specified volume of the specific controlled substance 28a, 28b, or 28c, the machine 20, system 50, and method may transfer the specified volume of the specific controlled substance from the filling station 38 to a dispensing location of the dispensing unit D, which dispenses the specified volume of the specific controlled substance to be sent to the patient. The specified volume is transferred through a sample conveyance system, which can be, but is not limited to, a conveyor belt or conveying system or mechanism (not shown). In one example, the specified volume of the specific controlled substance 28a, 28b, or 28c will be dispensed to a syringe or other comparable holding device or container 32. When the specified volume of the specific controlled substance is extracted and dispensed, the specified volume is subtracted from the current quantity volume of the specific controlled substance. Subsequently, the machine 20, system 50, network 52, and/or method may update the specific controlled substance with a new current quantity volume. Referring to the previous example, if the sub-compartment 24 and vessel 26 with Morphine initially held 100-ml, after extracting 5-ml, the new current quantity volume of the specific controlled substance is updated to be 95-ml.

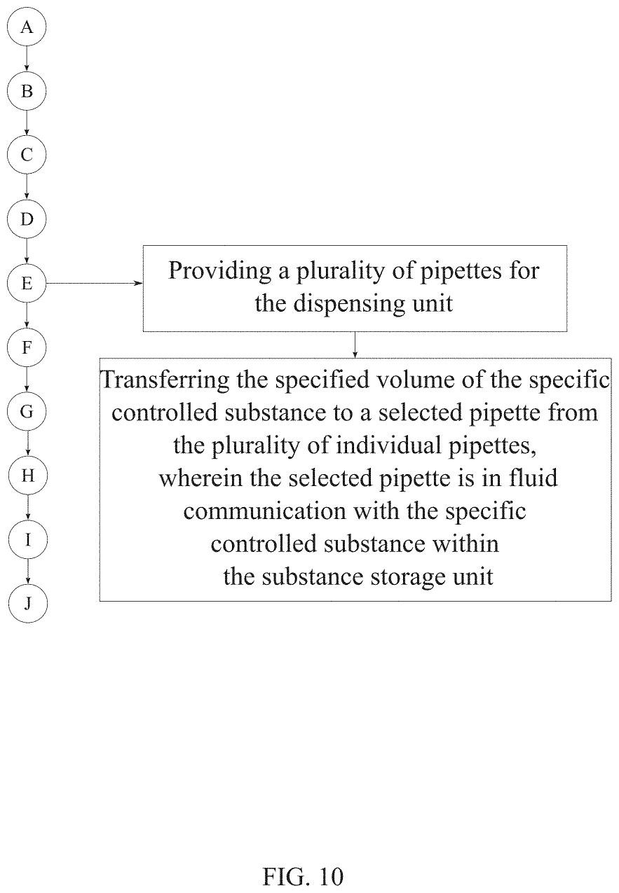

To accurately dispense the specified volume of the specific controlled substance 28a, 28b, or 28c, the dispensing unit D may be provided with the plurality of pipettes 40. As shown in FIGS. 1 and 10, each of the plurality of pipettes 40 is in fluid communication with a corresponding controlled substance from the plurality of controlled substances 28. For instance, if three controlled substances 28a, 28b, and 28c are stored in the substance storage unit S, the plurality of pipettes 40 will include three pipettes. Therefore, referring to the previous example, a selected pipette 40a, 40b, or 40c from the plurality of pipettes will be in fluid communication with the sub-compartment 24 and vessel 26 storing Morphine. As a result, the machine 20, system 50, and method may transfer the specified volume of the specific controlled substance to the selected pipette.

When the specified volume of the specific controlled substance 28a, 28b, or 28c is dispensed as required, the machine 20, system 50, and method may transfer the specified volume of the specific controlled substance to the labelling unit L through the dispensing unit. At the labelling unit L, the specified volume of the specific controlled unit is labelled with the at least one controlled substance data through a labelling mechanism. The container 32 holding the specified volume of the specific controlled substance will be labelled with information which can be, but is not limited to, medication name and medication quantity. Referring to the previous example, the specified volume of the specific controlled substance will be labelled as 5-ml of Morphine.

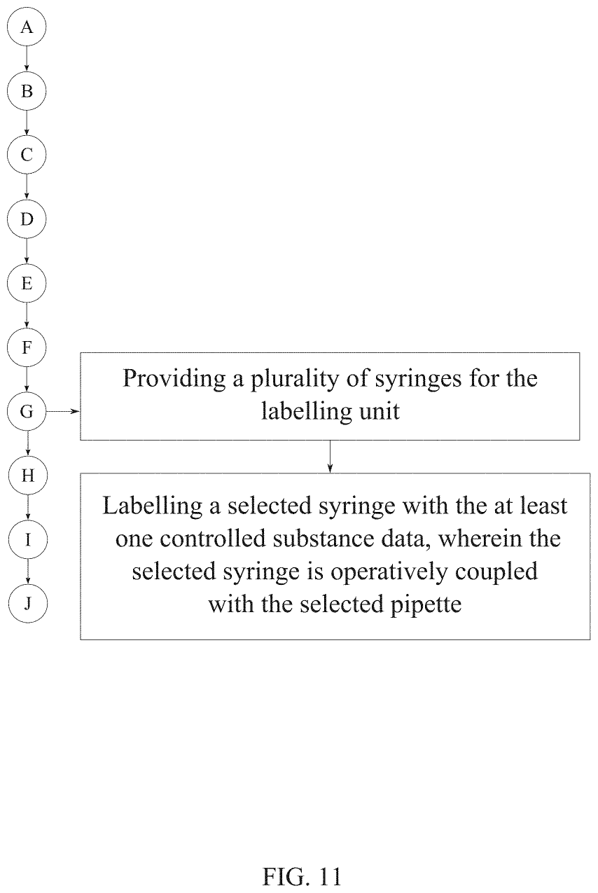

In one example, the labelling unit L may be provided with a plurality of syringes as the containers 32. As shown in FIG. 11, a selected syringe of the plurality of syringes is operatively coupled with a selected pipette from the plurality of pipettes 40. As a result, when the machine 20, system 50, and method transfers the specified volume of the specific controlled substance 28a, 28b, or 28c to the labelling unit L, the selected syringe is positioned adjacent the selected pipette 40, 40b, or 40c and filled. Therefore, with reference to the previous example, the selected syringe will be labelled as 5-ml Morphine. A mechanical arm or other comparable means (not shown) can be used to place the selected syringe adjacent the selected pipette.

When the labelling process is complete, a sample exiting system of the machine 20, system 50, and method may transfer the specified volume of the specific controlled substance to a distribution unit or retrieval station, which allows the medical professional to retrieve the container 32 holding the specified volume of the specific controlled substance 28a, 28b, 28c. The medical professional is granted access to the distribution unit through their authorized user account. The process may be repeated for each of the plurality of medical records, as needed or ordered. Since only prescribed amounts are distributed, amounts of the plurality of controlled substances are not wasted. Additionally, by having limited access, the overall misuse of the plurality of controlled substances is prevented. In one example, the distribution unit or retrieval station may include a sliding door or other comparable means that requires authentication prior to opening.

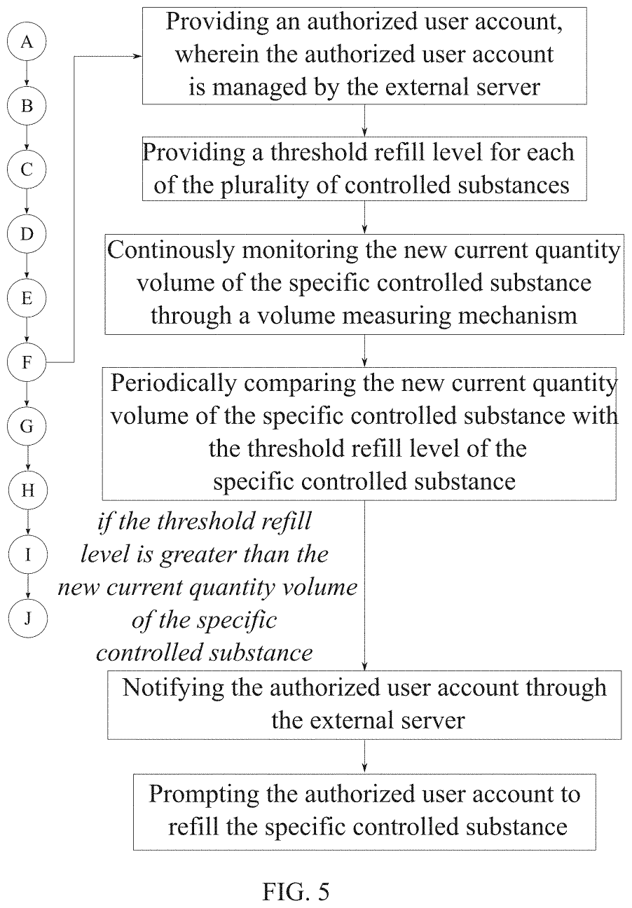

As discussed earlier, when the specified volume of the specific controlled substances is dispensed, the volume data of the plurality of controlled substances is updated with a new current quantity volume of the specific controlled substance. Continuous updating and monitoring of the new level of the specific controlled substance is important for refilling purposes. By doing so, the machine 20, system 50, network 52, and/or method may ensure that the specific controlled substance is always available for a patient. Continuously monitoring the new current quantity volume of the specific controlled substance through a volume measuring mechanism is also important to ensure that an excess amount of the specific controlled substance is not extracted through the machine 20, system 50, or method. As illustrated in FIG. 5, to refill the vessels 26 holding the specific controlled substances or any other arbitrary controlled substance of the plurality of controlled substances, the machine 20, system 50, network 52, and/or method may be provided with a threshold refill level for each of the plurality of controlled substances 28. While monitoring the new current quantity volume of the specific controlled substance, the machine 20, system 50, network 52, and/or method may periodically compare the new current quantity volume of the specific controlled substance with the threshold refill level of the specific controlled substance. The time interval for comparing can be predetermined by the authorized user account, the authorized user, the machine 20, the system 50, the network 52, and/or the method. As an example, if the time interval is 24-hours, the machine 20, system 50, network 52, and/or method may compare the new current quantity volume of the specific controlled substance with the threshold refill level of the specific controlled substance every 24-hours.

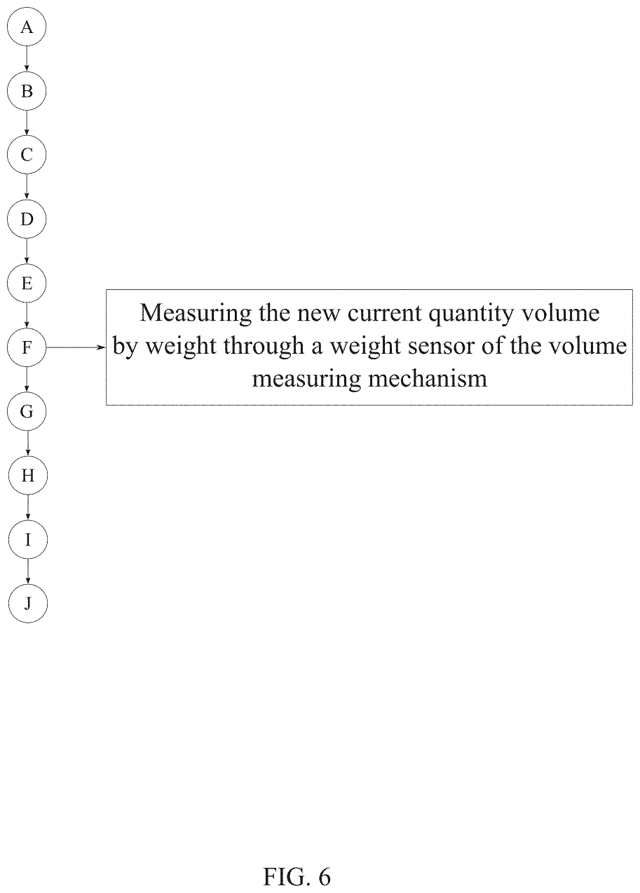

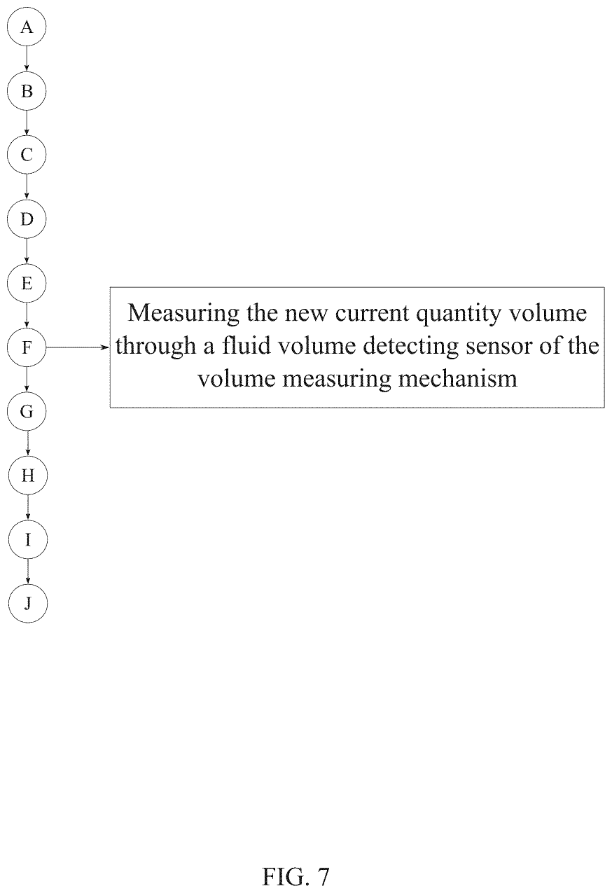

If the threshold refill level is greater than the new current quantity volume of the specific controlled substance, the machine 20, system 50, network 52, and/or method may notify the authorized user account or the authorized user through the external server, network, the machine, or the like. Next, the machine 20, system 50, network 52, and/or method may prompt the authorized user account or the authorized user to refill the specific controlled substance 28a, 28b, or 28c so that the specific controlled substance is available upon request. More specifically, the authorized user account or authorized user is prompted to refill the specific controlled substance such that the new current quantity volume is greater than the threshold refill level. Referring to the previous example wherein the specific controlled substance was Morphine, if the new level of the specific controlled substance is 45-ml, and the threshold refill level for Morphine is 50-ml, the authorized user account or authorized user is notified through the external server, the network, the machine, or the like. As illustrated in FIG. 6 and FIG. 7, the new current quantity volume of the specific controlled substance and the threshold refill level for each of the plurality of controlled substances 28 can be measured by volume or by weight. Sensors and other measuring mechanisms can be used to measure and monitor the new current quantity volume of the specific controlled substance and the threshold refill level of the plurality of controlled substances 28. More specifically, a weight sensor or a fluid volume detecting sensor (such as the sensors 36) of the volume measuring mechanism can be used.

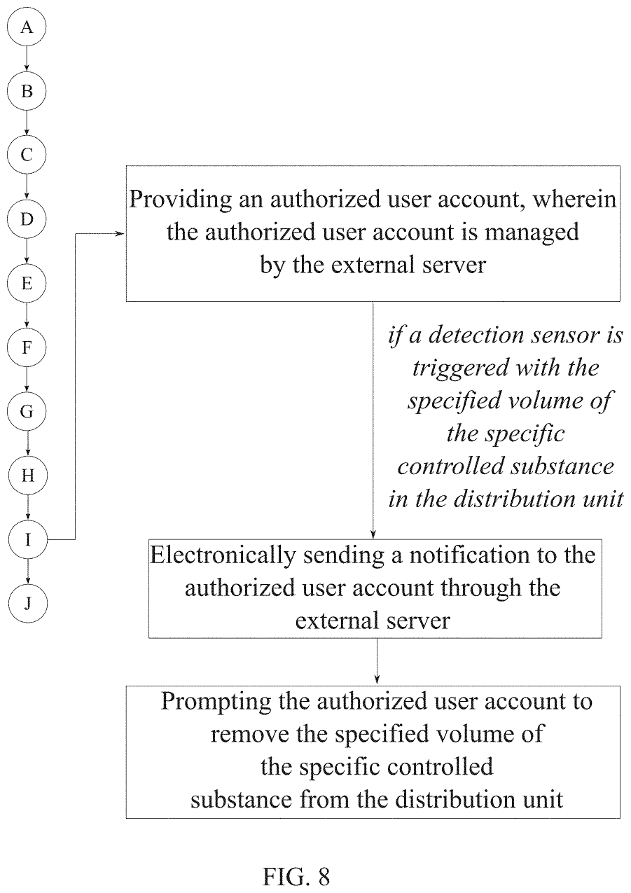

As described earlier, only the authorized user account or authorized user can access the substance storage unit S or the machine for refilling purposes. Similarly, for safety purposes, only the authorized user account or authorized user can access the distribution unit or retrieval station to retrieve the container 32 holding the specified volume of the specific controlled substance. As illustrated in FIG. 8, to retrieve the container 32 holding the specified volume of the specific controlled substance from the distribution unit, the machine 20, system 50, network 52, and/or method may electronically send a notification to the authorized user account or authorized user if a detection sensor is triggered with the container 32 holding the specified volume of the specific controlled substance in the distribution unit. After notifying the authorized user account or authorized user through the external server, the network, the machine, or the like, the authorized user account or authorized user may be prompted to remove the container 32 holding the specified volume of the specific controlled substance from the distribution unit. The authorized user account or authorized user can be granted access through a face recognition application, a fingerprint identification method, an access code, or other comparable authentication method.

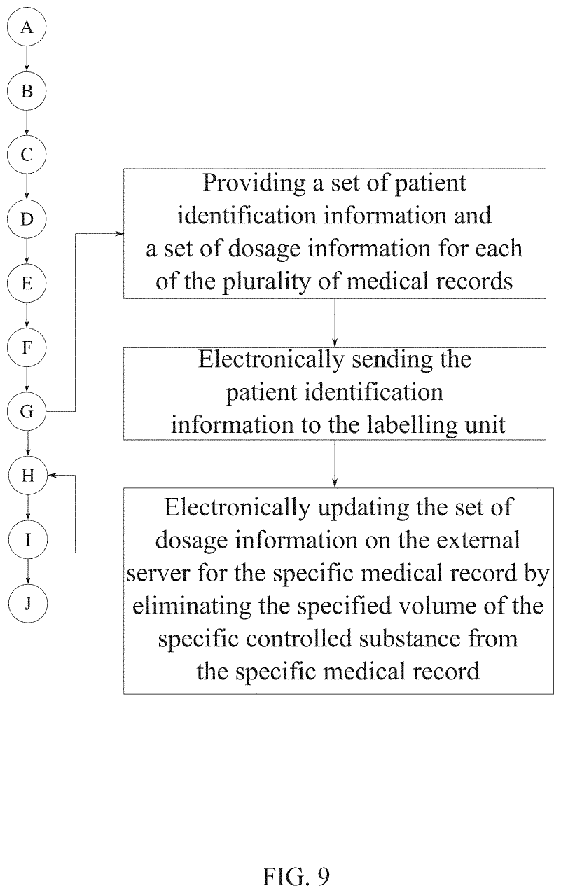

As described earlier, each of the plurality of medical records may comprise at least one controlled substance data. Referring to the previous example, the specific medical record contains information regarding Morphine. As shown in FIG. 9, in addition to the at least one controlled substance data, a set of patient identification information and a set of dosage information may be provided for each of the plurality of medical records. The set of patient identification information can be, but is not limited to, patient name, age, and address. The set of dosage information can contain information such as medication times and medication dosages for the specific patient. To ensure that the specified volume of the specific controlled substance is provided only to the intended patient, the machine 20, system 50, network 52, and/or method may electronically send the patient identification information to the labelling unit L. In the labelling unit L, the label 44, which can be, but is not limited to, a sticky label or other comparable method or device, is used to display the set of patient information on the selected syringe or other comparable container 32. When the specified volume of the specific controlled substance is extracted and available for distribution, the set of dosage information for the specific medical records is updated on the external server. More specifically, the machine 20, system 50, network 52, and/or method may electronically update the set of dosage information on the external server, the network, the machine, or the like, by eliminating the specified volume of the specific controlled substance from the specific medical record. Thus, duplicate orders are not processed for the specific medical record.

The machine 20, system 50, and method and components described herein can be configured using, or to be compatible with, various existing or to-be-developed electronic and computer technologies. The control device 34, i.e., a central processing unit (CPU) can include a set of instructions that can be executed to cause the CPU to perform any one or more of the methods or computer-based functions disclosed and described herein. The CPU may operate as a standalone control device or may be connected, e.g., using a network, to other computer systems or peripheral devices, either within the housing or shell 22 or remote from the housing. Any of the components discussed herein, such as the CPU, may be or emulate a computer system. The CPU may be specifically configured to implement various operations and functions for the machine 20, system 50, and method, and the various remote external components and devices of the system.

In a networked deployment, the CPU may operate in the capacity of a server or as a client user computer in a client-server user network environment, or as a peer computer system in a peer-to-peer (or distributed) network environment with one or more processors of the various components and devices of the black box system. Other of the components and devices of the machine 20, the system 50, and the method may include a separate processor, if desired. In one example, the CPU can be implemented with electronic devices that provide voice, video, or data communication. Further, while a single CPU is illustrated and described, the term “system” shall also be taken to include any collection of systems, sub-systems, or linked components and devices that individually or jointly execute a set, or multiple sets, of instructions to perform one or more computer functions.

The CPU may be or include one or more general processors, digital signal processors, specifically configured processors, application specific integrated circuits, field programmable gate arrays, servers, networks, digital circuits, analog circuits, combinations thereof, or other now known or later developed devices for analyzing and processing data. The CPU and other system processors may each implement a software program, such as code generated manually (i.e., programmed).

The CPU may include a memory that can communicate via a bus. The memory may be a main memory, a static memory, or a dynamic memory. The memory may include, but is not limited to, computer readable storage media such as various types of volatile and non-volatile storage media, including but not limited to random access memory, read-only memory, programmable read-only memory, electrically programmable read-only memory, electrically erasable read-only memory, flash memory, magnetic tape or disk, optical media, and the like. In one example, the memory can include a cache or random-access memory for the CPU. In alternative examples, the memory can be separate from the CPU, such as a cache memory of a processor, the system memory, or other memory. The memory may be an external storage device or database for storing data. Examples include a hard drive, compact disc (“CD”), digital video disc (“DVD”), memory card, memory stick, floppy disc, universal serial bus (“USB”) memory device, or any other device operative to store data. The memory is operable to store instructions executable by the CPU. The functions, acts or tasks described herein may be performed by the programmed CPU executing the instructions stored in the memory. The functions, acts or tasks are independent of the particular type of instruction set, storage media, processor or processing strategy, and may be performed by software, hardware, integrated circuits, firm-ware, micro-code, and the like, operating alone or in combination. Likewise, processing strategies may include multiprocessing, multitasking, parallel processing, and the like.

Additionally, the CPU may include an input device, such as the user interface 30, configured to allow a user to interact with any of the components of the CPU or the machine 20 and system 50. The input device may be a number pad, a keyboard, or a cursor control device, such as a mouse, or a joystick, touch screen display, wired or wireless remote control, or any other device operative to interact with the CPU.

The CPU may also include a disk or optical drive unit. The disk drive unit may include a computer-readable medium in which one or more sets of instructions, e.g., software, can be embedded. Further, the instructions may embody one or more of the methods, operations, functions, or logic as described herein. In one example, the instructions may reside completely, or at least partially, within the memory and/or within the processor during execution by the CPU. The memory and the processor also may include computer-readable media as discussed herein.

The present disclosure contemplates a computer-readable medium that includes instructions or receives and executes instructions responsive to a propagated signal, so that one or more components and devices connected to the system network can communicate voice, video, audio, images, or any other data over the system network. Further, the instructions may be transmitted or received over the network via a communication interface. The communication interface may be a part of the processor or CPU or may be a separate component. The communication interface may be created in software or may be a physical connection in hardware. The communication interface may be configured to connect with other remote external components and devices of the machine 20 and system 50 and/or with other internal components of the machine 20, system 50, or combinations thereof. The connection with the internal components may be a physical connection, such as a wired Ethernet connection or may be established wirelessly. Likewise, the additional connections with other external components and devices of the system may be physical connections or may be established wirelessly.

The machine 20 and system 50 network 52 may include wired networks, wireless networks, or combinations thereof. The wireless network may be a cellular telephone network, an 802.11, 802.16, 802.20, or WiMax network. Further, the machine 20 and system 50 network 52 should be a private network, such as an intranet, and may utilize a variety of networking protocols now available or later developed including, but not limited to, TCP/IP based networking protocols. Wireless signals may be RF signals and may be transmitted via antenna and transceivers within the system or may be transmitted via satellite.

Examples of the subject matter and the functional operations described in this specification can be implemented in digital electronic circuitry, or in computer software, firmware, or hardware, including the structures disclosed in this specification and their structural equivalents, or in combinations of one or more of them. Examples of the subject matter described in this specification can be implemented as one or more computer program products, i.e., one or more modules of computer program instructions encoded on a computer readable medium for execution by, or to control the operation of, data processing apparatus. While the computer-readable medium is shown to be a single medium, the term “computer-readable medium” includes a single medium or multiple media, such as a centralized or distributed database, and/or associated caches and servers that store one or more sets of instructions. The term “computer-readable medium” shall also include any medium that is capable of storing, encoding, or carrying a set of instructions for execution by a processor or that cause a computer system to perform any one or more of the methods or operations disclosed herein. The computer readable medium can be a machine-readable storage device, a machine-readable storage substrate, a memory device, or a combination of one or more of them. The term “data processing apparatus” encompasses all apparatus, devices, and machines for processing data, including by way of example a programmable processor, a computer, or multiple processors or computers. The apparatus can include, in addition to hardware, code that creates an execution environment for the computer program in question, e.g., code that constitutes processor firmware, a protocol stack, a database management system, an operating system, or a combination of one or more of them.

In a non-limiting example, the computer-readable medium can include a solid-state memory such as a memory card or other package that houses one or more non-volatile read-only memories. Further, the computer-readable medium can be a random-access memory or other volatile re-writable memory. Additionally, the computer-readable medium can include a magneto-optical or optical medium, such as a disk or tapes or other storage device to capture carrier wave signals such as a signal communicated over a transmission medium. A digital file attachment to an e-mail or other self-contained information archive or set of archives may be considered a distribution medium that is a tangible storage medium. Accordingly, the disclosure is considered to include any one or more of a computer-readable medium or a distribution medium and other equivalents and successor media, in which data or instructions may be stored.

In an alternative example, dedicated or otherwise specifically configured hardware implementations, such as application specific integrated circuits, programmable logic arrays and other hardware devices, can be constructed to implement one or more of the systems and methods described herein. Applications that may include the apparatus and systems of various embodiments can broadly include a variety of electronic and computer systems. One or more examples described herein may implement functions using two or more specific interconnected hardware modules or devices with related control and data signals that can be communicated between and through the modules, or as portions of an application-specific integrated circuit. Accordingly, the present system encompasses software, firmware, and hardware implementations.

In accordance with various embodiments of the present disclosure, the methods described herein may be implemented by software programs executable by a computer system. Further, in an exemplary, non-limited embodiment, implementations can include distributed processing, component/object distributed processing, and parallel processing. Alternatively, virtual computer system processing can be constructed to implement one or more of the methods or functionality as described herein.

Although the present specification describes components and functions that may be implemented in particular examples with reference to particular standards and protocols, the invention is not limited to such standards and protocols. For example, standards for Internet and other packet switched network transmission (e.g., TCP/IP, UDP/IP, HTML, HTTP, HTTPS) represent examples of the state of the art. Such standards are periodically superseded by faster or more efficient equivalents having essentially the same functions. Accordingly, replacement standards and protocols having the same or similar functions as those disclosed herein are considered equivalents thereof.

A computer program (also known as a program, software, software application, script, or code) can be written in any form of programming language, including compiled or interpreted languages, and it can be deployed in any form, including as a standalone program or as a module, component, subroutine, or other unit suitable for use in a computing environment. A computer program does not necessarily correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

The processes and logic flows described in this specification can be performed by one or more programmable processors executing one or more computer programs to perform functions by operating on input data and generating output. The processes and logic flows can also be performed by, and apparatus can also be implemented as, special purpose logic circuitry, e.g., an FPGA (field programmable gate array) or an ASIC (application specific integrated circuit).

Processors suitable for the execution of a computer program include, by way of example, both general and special purpose microprocessors, and anyone or more processors of any kind of digital computer. Generally, a processor will receive instructions and data from a read only memory or a random-access memory or both. The essential elements of a CPU may be a processor for performing instructions and one or more memory devices for storing instructions and data. Generally, The CPU may also include, or be operatively coupled to receive data from or transfer data to, or both, one or more mass storage devices for storing data, e.g., magnetic, magneto optical disks, or optical disks. However, the CPU need not have such devices. Moreover, the CPU can be embedded in another device or component of the machine 20 or system 50. Computer readable media suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto optical disks; and CD ROM and DVD-ROM disks. The processor and the memory can be supplemented by, or incorporated in, special purpose logic circuitry.

As used herein, the terms “microprocessor” or “general-purpose processor” (“GPP”) may refer to a hardware device that fetches instructions and data from a memory or storage device and executes those instructions (for example, an Intel Xeon processor or an AMD Opteron processor) to then, for example, process the data in accordance therewith. The term “reconfigurable logic” may refer to any logic technology whose form and function can be significantly altered (i.e., reconfigured) in the field post-manufacture as opposed to a microprocessor, whose function can change post-manufacture, e.g. via computer executable software code, but whose form, e.g. the arrangement/layout and interconnection of logical structures, is fixed at manufacture. The term “software” may refer to data processing functionality that is deployed on a GPP. The term “firmware” may refer to data processing functionality that is deployed on reconfigurable logic. One example of a reconfigurable logic is a field programmable gate array (“FPGA”) which is a reconfigurable integrated circuit. An FPGA may contain programmable logic components called “logic blocks”, and a hierarchy of reconfigurable interconnects that allow the blocks to be “wired together”, somewhat like many (changeable) logic gates that can be inter-wired in (many) different configurations. Logic blocks may be configured to perform complex combinatorial functions, or merely simple logic gates like AND, OR, NOT and XOR. An FPGA may further include memory elements, which may be simple flip-flops or more complete blocks of memory.

To provide for interaction with an authorized user, examples of the subject matter described in this specification can be downloaded or uploaded to another remote computer or electronic device from the machine 20 and/or system 50. Such a remote device can then utilize a display, e.g., a CRT (cathode ray tube) or LCD (liquid crystal display) monitor, for displaying information to the user and a keyboard and a pointing device, e.g., a mouse or a trackball, by which the user can provide input to the remote computer or electronic device. Other kinds of remote devices can be used to provide for interaction with a user as well. Feedback provided to the user from the remote electronic device or computer can be any form of sensory feedback, e.g., visual feedback, auditory feedback, or tactile feedback. Input from the user to the remote electronic device or computer can be received in any form, including acoustic, speech, or tactile input.

Although certain machines, methods, and systems for dispensing and monitoring controlled substances have been described herein in accordance with the teachings of the present disclosure, the scope of coverage of this patent is not limited thereto. On the contrary, this patent covers all embodiments of the teachings of the disclosure that fairly fall within the scope of permissible equivalents.

What is claimed is:

1. A method of monitoring and dispensing controlled substances, the method comprising the steps of:
providing a machine containing a plurality of controlled substances, each of the plurality of controlled substances stored in the machine in separate vessels, the machine connected with a network having an accessible patient information database;
receiving, by the machine from the network, an order for a specific patient having a specific patient record in the patient information database for a prescribed amount of one controlled substance of the plurality of controlled substances;
dispensing only the prescribed amount of the one controlled substance to a container from a respective vessel of the separate vessels;
permitting an authorized user to retrieve the container having the dispensed prescribed amount of the one controlled substance from the machine to fill the order; and
repeating each of the steps when a new order is received by the machine from the network for any one of the plurality of controlled substances.

2. The method of claim 1, wherein the step of dispensing includes dispensing the prescribed amount of the one controlled substance into a syringe as the container.

3. The method of claim 1, wherein the step of providing includes providing the machine having a control device with a processor and a memory, the control device configured to determine a current volume of the one controlled substance before the step of dispensing and to determine a new current volume of the one controlled substance after the step of dispensing, the new current volume becoming the current volume before a subsequent step of dispensing the one controlled substance.

4. The method of claim 3, further comprising the steps of:
storing a threshold refill level for a corresponding substance of the plurality of controlled substances for each respective vessel of the separate vessels;
monitoring the new current volume of each of the separate vessels;
comparing the new current volume of each respective vessel of the separate vessels to the respective threshold refill level for each corresponding substance of the plurality of controlled substances;
notifying an authorized user if the new current volume is less than the threshold refill level for any one vessel of the separate vessels; and
prompting the notified authorized user to refill the one vessel with the corresponding substance.

5. The method of claim 1, further comprising the step of including an authorized user account for the authorized user, wherein the authorized user account is managed by an external server of the network.

6. The method of claim 1, wherein the step of dispensing further includes transferring the prescribed amount of the one controlled substance from the respective vessel to the container in a filling station of the dispensing unit.

7. The method of claim 6, wherein the filling station includes a plurality of pipettes, each arranged to cooperate with one of the separate vessels for transferring a corresponding one of the plurality of controlled substances.

8. The method of claim 1, further comprising the step of applying a label to the container, the label including patient information that at least identifies the specific patient.

9. The method of claim 8, wherein the step of applying further includes the label having substance information that at least identifies the one controlled substance and the prescribed amount.

10. The method of claim 1, wherein each of the plurality of controlled substances is selected from a group consisting of Hydromorphone, Methadone, Meperidine, Oxycodone, Morphine, Fentanyl, and Codeine.

11. The method of claim 1, further comprising the steps of:
sending a notification to the authorized user through the network or the machine when the order is received by the machine; and
prompting the authorized user to remove the container having the prescribed amount of the one controlled substance from the machine.

12. The method of claim 1, wherein the specific patient record includes patient identification information, prescribed controlled substance information, and dosage information for each prescribed controlled substance, and wherein the method further comprises:
sending the patient identification information, prescribed controlled substance information, and dosage information of the specific patient record to the machine; and
updating the dosage information for the specific patient record after the one controlled substance is dispensed.

13. A system for monitoring and dispensing controlled substances, the system comprising:
a machine containing a plurality of controlled substances, each of the plurality of controlled substances stored in the machine in separate vessels;
a network connected to the machine, the network having an accessible patient information database;
the machine configured to receive, from the network, an order for a specific patient from the patient information database for a prescribed amount of one controlled substance of the plurality of controlled substances;
the machine configured to, when the order is received, dispense only the prescribed amount of the one controlled substance to a container from a corresponding vessel of the separate vessels; and
a user interface of the machine permitting an authorized user to obtain access to and retrieve the container having the dispensed specific amount of the one controlled substance from the machine.

14. The system of claim 13, wherein the prescribed amount of the one controlled substance is dispensed into a syringe as the container.

15. The system of claim 13, wherein the machine has a control device with a processor and a memory, the control device being configured to determine a current volume of the one controlled substance before dispensing any of the one controlled substance, and to determine a new current volume of the one controlled substance after dispensing, the new current volume becoming the current volume before a next subsequent dispensing of the one controlled substance.

16. The system of claim 13, wherein the separate vessels are refillable, replaceable, or refillable and replaceable within the machine, and wherein the network or the machine can detect when the new current level of each of the plurality of controlled substances reaches a threshold level within the corresponding separate vessels.

17. The system of claim 16, wherein the machine or the network notifies an authorized user if the new current volume is less than the threshold refill level for any one vessel of the separate vessels, and/or prompts the notified authorized user to refill the one vessel with the corresponding substance.

18. The system of claim 13, wherein the machine transfers the prescribed amount of the one controlled substance from the respective vessel to the container in a filling station of the dispensing unit, and wherein the filling station includes a plurality of pipettes, each arranged to cooperate with one of the separate vessels for transferring a corresponding one of the plurality of controlled substances.

19. The system of claim 13, wherein the machine applies a label to the container, the label including patient information that at least identifies the specific patient and at least identifies the one controlled substance and the prescribed amount.

20. The system of claim 12, wherein each of the plurality of controlled substances is selected from a group consisting of Hydromorphone, Methadone, Meperidine, Oxycodone, Morphine, Fentanyl, and Codeine.
